(19) United States
(12) Patent Application Publication
(10) Pub. No.: US 2013/0143844 A1
(43) Pub. Date: Jun. 6, 2013

(54) ECTOPARASITICIDE-CONTAINING PREPARATION FOR SPONTANEOUS EMULSION FORMATION

(71) Applicants: Dirk Mertin, Langenfeld (DE); Bernd Bigalke, Bergisch Gladbach (DE)

(72) Inventors: Dirk Mertin, Langenfeld (DE); Bernd Bigalke, Bergisch Gladbach (DE)

(73) Assignee: BAYER ANIMAL HELATH GMBH, Monheim (DE)

(21) Appl. No.: 13/805,610

(22) PCT Filed: Jun. 15, 2011
(86) PCT No.: PCT/EP11/59895

§ 371 Date: Jan. 17, 2013

(30) Foreign Application Priority Data
Jun. 19, 2010 (EP) 10006385.8

(51) Int. Cl. A01N 53/00; A61K 9/107; A01N 57/20

(52) U.S. Cl. CPC A61K 9/1075
USPC 514/100; 514/521

(57) ABSTRACT

An emulsion concentrate for spontaneous emulsion preparation is described, comprising at least one ectoparasiticidal active ingredient. Moreover, a process for the preparation of a corresponding emulsion concentrate, and also its use for producing emulsions are described. An emulsion which is obtained starting from the emulsion concentrate, and also its use, are also described.

The present invention relates to an emulsion concentrate for spontaneous emulsion preparation, comprising at least one ectoparasiticide. The present invention further relates to a process for the preparation of a corresponding emulsion concentrate, and also to its use for producing emulsions. The present invention also relates to an emulsion which is obtained starting from the emulsion concentrate, and also to its use.

Emulsion concentrates (EC) are generally liquid, homogeneous preparations which, after being introduced into water, spontaneously form emulsions, without the need for a high dispersion energy. Besides the active ingredient, emulsion concentrates of this type optionally also comprise a solvent as well as an appropriate dispersant and are used in the field of crop protection, farm hygiene and veterinary medicine.

Emulsion concentrates generally comprise lipophilic active ingredients which are dissolved in nonpolar solvents. The most important solvents for emulsion concentrates are nonpolar hydrocarbons, for example xylene, paraffin, vegetable oils, naphtha, Solvesso or Shellsol. However, it is also possible to use other solvents with limited miscibility in water, for example methyl isobutyl ketone (MIBK). The selection of solvents is determined inter alia by solubility of the active ingredients, cost, toxicity, viscosity and density.

Further constituents of emulsion concentrates are emulsifiers and coemulsifiers. Suitable emulsifiers ensure, together with the further constituents, that the emulsion concentrate is spontaneously dispersible in water, i.e. without the input of high energy, and forms a stable emulsion therein. Parameters for assessing emulsifying power are initial spontaneity, sedimentation, stability towards temperature and water hardness, electrolyte tolerance and particle size. Optimal product properties are sometimes also achieved by a mixture of a nonionic and an anionic emulsifier. The anionic emulsifiers used are usually alkylarylsulphonates and salts thereof with alkaline earth metal ions or amines and alkylphenol-, polyacrylaryl- or alkylethoxylated phosphate esters. Ca dodecylbenzenesulphonate is an example and is commercially available, for example, under the names Arylan® CA, Ca DBS 50 SA, Casul® 70 HF, Emcol® P1020B, Emulson® AG/CAL, Ifracide® LCAB, Phenylsulfonat CA, Rhodacal®.

Sermul® EA88 or Witconate®, is commercially available and is by far the most-used surfactant of this class. Nonionic emulsifiers which are used are, for example, polyethylene glycol (PEG) fatty alcohol ethers, PEG alkylphenol ethers, PEG alkylphenol resins, ethoxylated vegetable oils, PEG fatty acid esters, ethoxylated fatty amines, sorbitans and ethoxylated sorbitans (polysorbates). Mixtures of anionic and nonionic emulsifiers, which are in most cases dissolved in suitable solvents, are likewise supplied commercially (Berol®, Sponto® and Emulsogen®).

Coemulsifiers are sometimes required if it is not possible to attain an adequate stability in hard water through selection of the ratio of the nonionic and anionic emulsifier component. Ethylene oxide/propylene oxide block polymers (poloxamers) are often used for this purpose.

However, the use of the aforementioned anionic emulsifiers is subject to certain restrictions in the field of veterinary medicine. Medicaments for food-producing animals which are intended for sale in the European community have to be investigated with regard to the residues of the medicaments and their degradation products in the foods. Depending on the toxicity and the average uptake amount of the food, so-called maximum residue limits (MRL) are stipulated for the active ingredients and auxiliaries used as feed materials by the European Medicines Agency (EMEA). Certain auxiliaries are explicitly excluded from the establishment of an MRL value. However, the alkylbenzenesulphonates suitable in the prior art for formulating emulsion concentrates are only provisionally freed from the establishment of a maximum residue limit in the case of cattle and sheep. For use in other livestock groups, for example poultry and pigs, these emulsifiers are not available without establishment of a maximum residue limit. However, the determination of a maximum residue limit requires complex and cost-intensive toxicological studies, as a result of which ultimately the usability of an auxiliary is prevented and/or the costs required for registration are so high that the users do not carry out registration. Consequently, for use in the field of veterinary medicine, emulsifiers and coemulsifiers are desired which have either already been registered and have corresponding maximum residue limits, or else are not subject to a registration requirement.

Anionic emulsifiers already provided with a maximum residue limit and thus listed, such as ammonium lauryl sulphate, sodium cetostearyl sulphate, sodium lauryl sulphate and sodium stearate, are not very suitable, on account of their low solubility in lipophilic solvents, for forming corresponding emulsion concentrates.

Accordingly, the object of the present invention is to provide an emulsion concentrate for insecticidally active, acaricidally active ectoparasiticides. The emulsifiers or coemulsifiers to be used in these emulsion concentrates should preferably be those which, according to current knowledge, either already have a registration for use in the field of medicaments for food-producing animals by the European Medicines Agency, or else are excluded from the establishment of a corresponding MRL value. Moreover, the emulsions which are formed upon dilution with water should be (physically) stable.

This object is achieved by a composition comprising an ectoparasiticidal active ingredient, at least one anionic surfactant, a nonionic surfactant and an organic solvent.

It has been found that the emulsion concentrates according to the invention of ectoparasiticides have adequate stability and, as a result of the selection of suitable material systems, can comprise emulsifiers and/or coemulsifiers which already have a registration for use in the field of medicaments for food-producing animals by the European Medicines Agency, or else are excluded from the establishment of a corresponding MRL value. Consequently, the use of the composition according to the invention in the field of veterinary medicine is possible without restrictions. In particular, the use of the composition according to the invention for the veterinary treatments of cattle, sheep, poultry and pigs is possible.

The individual constituents of the emulsion concentrate are described in more detail below:

### (1) Active Ingredient

As active ingredient, it is possible to use substances which have a paralyzing or killing effect on parasites which live on or in the skin of animals.

The active ingredients include insecticides such as phosphorus-containing compounds, i.e. phosphoric or phosphonic acid esters, natural or synthetic pyrethroids, carbamates, amidines, juvenile hormones and juvenoid synthetic active ingredients, and also chitin synthesis inhibitors such as diaryl ethers, benzoylureas and triazines.

The phosphoric or phosphonic acid esters include:
O-ethyl O-(8-quinolyl)phenyl-thiophosphate (quintiofos),
O,O-diethyl O-(3-chlor-4-methyl-7-coumarinyl)thiophosphate (coumaphos),
O,O-diethyl O-phenylglyoxylonitrile oxime thiophosphate (phoxim),
O,O-diethyl O-cyanochlorobenzaldoxime thiophosphate (chlorphoxim),
O,O-diethyl O-(4-bromo-2,5-dichlorophenyl)phosphorothionate (bromophos-ethyl),
O,O,O′,O′-tetraethyl S,S′-methylenedi(phosphorodithionate) (ethion),
2,3-p-dioxanedithiol S,S-bis(O,O-diethylphosphorodithionate),
2-chloro-1-(2,4-dichlorophenyl)vinyl diethyl phosphate (chlorfenvinphos),
O,O-dimethyl O-(3-methyl-4-methylthiophenyl)thionophosphoric acid ester (fenthion).

The carbamates include:
2-isopropoxyphenyl methylcarbamate (propoxur),
1-naphthyl N-methylcarbamate (carbaryl).

The synthetic pyrethroids include
(α-cyano-4-fluoro-3-phenoxy)benzyl 3-[2-(4-chlorophenyl)-2-chlorovinyl]-2,2-dimethylcyclopropenecarboxylate (flumethrin),
α-cyano-(4-fluoro-3-phenoxy)benzyl 2,2-dimethyl-3-(2,2-dichlorovinyl)cyclopropanecarboxylate (cyfluthrin) and its enantiomers and stereomers.
α-cyano-3-phenoxybenzyl (±)-cis,trans-3-(2,2-dibromovinyl)-2,2-dimethylcyclopropanecarboxylate (deltamethrin),
α-cyano-3-phenoxybenzyl 2,2-dimethyl-3-(2,2-dichlorovinyl)cyclopropanecarboxylate (cypermethrin),
3-phenoxybenzyl (±)-cis,trans-2,2-dichlorovinyl-2,2-dimethylcyclopropanecarboxylate (permethrin),
α-cyano-3-phenoxybenzyl α-(p-Cl-phenyl)isovalerate (fenvalerate),
2-cyano-3-phenoxybenzyl 2-(2-chloro-α,α,α-trifluoro-p-toluidino)-3-methylbutyrate (fluvalinate).

The amidines include:
3-methyl-2-[2,4-dimethylphenylimino]thiazoline,
2-(4-chloro-2-methylphenylimino)-3-methylthiazolidine,
2-(4-chloro-2-methylphenylimino)-3-(isobutyl-1-enyl)thiazolidine
1,5-bis(2,4-dimethylphenyl)-3-methyl-1,3,5-triazapenta-1,4-diene (amitraz).

Cyclic macrolides such as ivermectins and abamectins. In this regard, mention may be made for example of 5-O-dimethyl-22,23-dihydroavermectin-A₁ₐ, -22,23-dihydroavermectin B₁ₐ and 22,23-dihydroavermectin B_{b1} (cf. for example WHO. F.A. Series 27, pp. 27-73 (1991)).

The juvenile hormones and juvenile-hormone-like substances include in particular compounds of the following formulae:

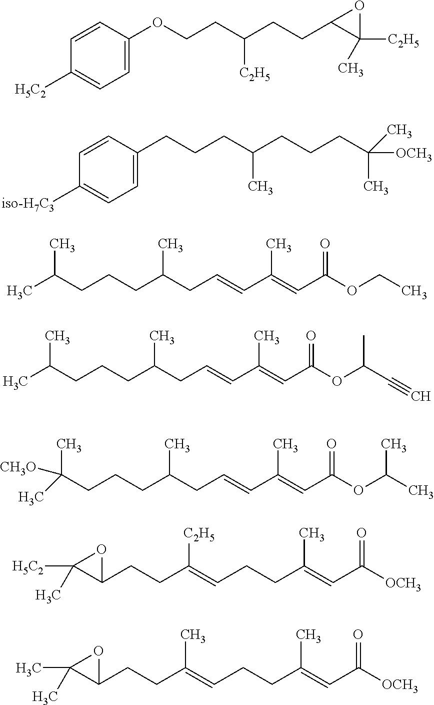

The substituted diaryl ethers include in particular

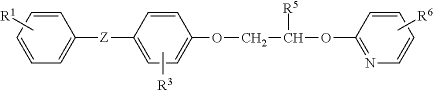

| R¹ | R³ | R⁵ | R⁶ | Z |
|---|---|---|---|---|
| H | H | CH₃ | H | O |
| H | H | CH₃ | 2-Cl | O |
| 5-F | H | CH₃ | H | O |
| H | H | CF₃ | H | O |
| H | H | C₂H₅ | H | O |
| H | H | H | H | O |
| H | H | CH₃ | H | CH₂ |
| H | H | CH₃ | H | C(CH₃)2 |

The benzoylureas include compounds of the formula

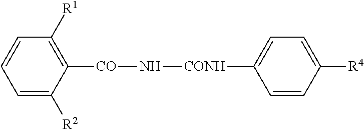

| R¹ | R² | R⁴ |
|---|---|---|
| H | Cl | CF₃ |
| Cl | Cl | CF₃ |
| F | F | CF₃ |
| H | F | CF₃ |
| H | Cl | SCF₃ |
| F | F | SCF₃ |
| H | F | SCF₃ |
| H | Cl | OCF₃ |
| F | F | OCF₃ |
| H | F | OCF₃ |
| F | F | 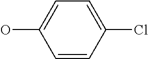 |
| F | F | 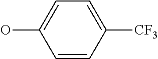 |
| F | F | 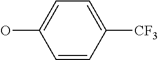 |

The triazines include compounds of the formula

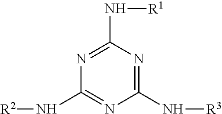

| R¹ | R² | R³ |
|---|---|---|
| Cyclopropyl | H | H |
| Cyclopropyl | H | CH₃ |
| Cyclopropyl | H | C₂H₅ |
| Cyclopropyl | H | C₃H₇-n |
| Cyclopropyl | H | C₄H₉-n |
| Cyclopropyl | H | C₅H₁₁-n |
| Cyclopropyl | H | C₆H₁₃-n |
| Cyclopropyl | H | C₇H₁₅-n |
| Cyclopropyl | H | C₈H₁₇-n |
| Cyclopropyl | H | C₁₂—H₂₅-n |
| Cyclopropyl | H | CH₂—C₄H₉-n |
| Cyclopropyl | H | CH₂CH(CH₃)C₂H₅ |
| Cyclopropyl | H | CH₂CH═CH₂ |
| Cyclopropyl | Cl | C₂H₅ |
| Cyclopropyl | Cl | C₆H₁₃-n |
| Cyclopropyl | Cl | C₈H₁₇-n |
| Cyclopropyl | Cl | C₁₂H₂₅-n |
| Cyclopropyl | H | Cyclopropyl |
| Cyclopropyl | H | COCH₃ |
| Cyclopropyl | H | COCH₃ HCl |
| Cyclopropyl | H | COC₂H₅ HCl |
| Cyclopropyl | H | COC₂H₅ |
| Cyclopropyl | H | COC₃H₇-n |
| Cyclopropyl | H | COC₃H₇-i |
| Cyclopropyl | H | COC₄H₉-t HCl |
| Cyclopropyl | H | COC₄H₉-n |
| Cyclopropyl | H | COC₆H₁₃-n |
| Cyclopropyl | H | COC₁₁—H₂₃-n |
| Cyclopropyl | COCH₃ | COC₂H₅ |
| Cyclopropyl | COC₃H₇-n | COC₆H₁₃-n |
| Cyclopropyl | COCH₃ | COC₃H₇-n |
| Cyclopropyl | COC₂H₅ | COC₃H₇-n |
| Cyclopropyl | H | COCyclopropyl |
| Cyclopropyl | COCyclopropyl | COCyclopropyl |
| Cyclopropyl | COCH₃ | COCH₃ |
| Isopropyl | H | H |
| Isopropyl | H | COCH₃ |
| Isopropyl | H | COC₃H₇-n |
| Cyclopropyl | H | CONHCH₃ |
| Cyclopropyl | H | CONHC₃H₇-i |
| Cyclopropyl | CONHCH₃ | CONHCH₃ |
| Cyclopropyl | H | SCNHCH₃ |
| Cyclopropyl | H | CONHCH₂CH═CH₂ |
| Cyclopropyl | CONHCH₂CH═CH₂ | CONHCH₂CH═CH₂ |
| Cyclopropyl | CSNHCH₃ | CSNHCH₃ |

Particular emphasis may be given to the active ingredients propoxur, flumethrin, permethrin, cyfluthrin, amitraz, coumaphos, imidacloprid, abamectin, ivermectin, pyriproxyfen, phoxim, and fenthion.

The composition according to the invention comprises the ectoparasiticidal active ingredient preferably in an amount of from 1 to 75% by weight, particularly preferably from 2 to 50% by weight, in particular from 5 to 20% by weight.

### (2) Anionic Surfactant

The composition according to the invention further comprises at least one anionic surfactant.

The anionic surfactant is preferably selected from the group of sulphosuccinates, alkylaryl sulphates, alkylarylsulphonates, and also their salts.

Within the context of the present invention, it is further preferred if the anionic surfactant is a surfactant which already has a registration for use in the field of medicaments for food-producing animals by the European Medicines Agency or else is excluded from the establishment of a corresponding MRL value according to current knowledge.

Examples of corresponding anionic surfactants are sodium dodecyl sulphate, sodium dioctyl sulphosuccinate, calcium dodecyl sulphate and calcium dioctyl sulphosuccinate.

The composition according to the invention comprises the anionic surfactant preferably in an amount of from 1 to 30% by weight, particularly preferably from 2 to 20% by weight, in particular from 10 to 15% by weight, in each case based on the composition.

### (3) Nonionic Surfactants

Nonionic surfactants are, for example, alkanolamides, polycondensates of ethylene oxide with fatty alcohols, fatty acids, fatty amines, mono-, di-, triglycerides or substituted phenols, block copolymers with ethoxy and propoxy groups (ethylene oxide/propylene oxide block polymers (polaxamers)); esters of fatty acids with polyols such as glycerol or glycol, organopolysiloxanes, sorbitan derivatives (polysorbates, sorbitan esters), ethers or esters of sucrose or glucose. Preference is given to using polysorbates, ethoxylated hydroxy fatty acids, ethoxylated castor oil or ethoxylated hydrogenated castor oil.

The composition according to the invention comprises the nonionic surfactant preferably in an amount of from 1 to 30% by weight, particularly preferably from 2 to 20% by weight, in particular from 10 to 20% by weight, in each case based on the composition.

### (4) Organic Solvent

The composition according to the invention moreover comprises at least one organic solvent.

The organic solvent is preferably selected from the group consisting of the group of benzoic acid esters and the limited-water-miscibility ketones.

Solvents from the class of benzoic acid esters are, for example, methyl benzoate (benzoic acid methyl ester), ethyl benzoate (benzoic acid ethyl ester), propyl benzoate (benzoic acid propyl ester), isopropyl benzoate (benzoic acid isopropyl ester), isoamyl benzoate (benzoic acid isoamyl ester), butyl benzoate (benzoic acid butyl ester), tert-butyl benzoate (benzoic acid tert-butyl ester) and benzyl benzoate (benzoic acid benzyl ester). Preference is given to using methyl benzoate and benzyl benzoate.

Solvents from the class of limited-water-miscibility ketones are, for example, methyl isobutyl ketone (4-methyl-2-pentanone), methyl phenyl ketone (acetophenone), methyl 1-naphthyl ketone (1-acetylnaphthalene), 1-chloroethyl methyl ketone (3-chloro-2-butanone), methyl octyl ketone (2-decanone), dicyclohexyl ketone, dicyclopropyl ketone, diethyl ketone (3-pentanone), diisobutyl ketone (2,6-dimethyl-4-heptanone), tert-butyl methyl ketone (3,3-dimethyl-2-butanone), ethyl methyl ketone (2-butanone), methyl pentyl ketone (2-heptanone), ethyl butyl ketone (3-heptanone), dipropyl ketone (4-heptanone), propyl diketone (2,3-hexanedione), ethyl propyl ketone (3-hexanone), methyl isopropyl ketone (3-methyl-2-butanone), isopropyl phenyl ketone, ethyl isoamyl ketone (5-methyl-3-heptanone), methyl isohexenyl ketone (6-methyl-5-hepten-2-one), methyl isoamyl ketone (5-methyl-2-hexanone), methyl propyl ketone (2-pentanone), methyl heptyl ketone (2-nonanone), ethyl hexyl ketone (3-nonanone), dibutyl ketone (5-nonanone), methyl hexyl ketone (2-octanone), ethyl pentyl ketone (3-octanone), ethyl phenyl ketone (propiophenone), methyl nonyl ketone (2-undecanone). Preference is given to using methyl isobutyl ketone.

The composition according to the invention comprises the at least one organic solvent preferably in an amount of from 10 to 90% by weight, particularly preferably from 20 to 80% by weight, in particular from 30 to 70% by weight, in each case based on the composition.

Despite the considerably different polarity and solvating ability of the benzoic acid esters and lipophilic ketones, it has surprisingly been found that these, with a mixture of a sulphosuccinate and a nonionic surfactant, form emulsion concentrates which can be easily dispersed in water and form stable emulsions therein.

### (5) Further Constituents

Moreover, the composition according to the invention can have further optional constituents.

### (5.1) Cosolvents

The composition according to the invention can moreover also comprise at least one cosolvent.

In cases where the composition according to the invention comprises a cosolvent, this cosolvent can preferably be selected from the group consisting of water; alkanols; glycols; polyethylene glycols; polypropylene glycols; glycerol; aromatic alcohols, such as benzyl alcohol, phenylethanol, phenoxyethanol; esters, such as ethyl acetate, butyl acetate, ethers, such as alkylene glycol alkyl ethers, such as dipropylene glycol monomethyl ether, diethylene glycol monobutyl ether; ketones, such as acetone, methyl ethyl ketone; aromatic and/or aliphatic hydrocarbons, vegetable or synthetic oils, DMF, dimethylacetamide, N-methylpyrrolidone and 2-dimethyl-4-oxymethylene-1,3-dioxolane.

If the composition according to the invention comprises the at least one cosolvent, then this cosolvent can preferably be present in an amount of from 1 to 50% by weight, particularly preferably from 2 to 30% by weight, in particular from 5 to 30% by weight, in each case based on the composition.

### (5.2) Thickeners

The composition according to the invention can moreover also comprise at least one thickener.

Thickeners are, for example, inorganic thickeners, such as bentonites, colloidal silica, aluminium monostearate, organic thickeners, such as cellulose derivatives, polyvinyl alcohols, polyvinylpyrrolidones and copolymers thereof, acrylates and methacrylates.

If the composition according to the invention comprises the at least one thickener, then this thickener can preferably be present in an amount of from 0.1 to 10% by weight, particularly preferably from 0.5 to 10% by weight, in particular from 1 to 10% by weight, in each case based on the composition.

### (5.3) Dyes

The composition according to the invention can moreover also comprise at least one dye.

Dyes which may be mentioned are all dyes which are approved for use on animals, which may be dissolved or suspended, such as, for example, anthraquinone dyes, azo dyes, dioxazine dyes, nitro and nitroso dyes, phthalocyanine dyes, sulphine dyes or triphenylmethane dyes.

If the composition according to the invention comprises the at least one dye, then this dye can preferably be present in an amount of from 0.01 to 5% by weight, particularly preferably from 0.1 to 2% by weight, in particular from 0.1 to 1% by weight, in each case based on the composition.

### (5.4) Further Auxiliaries

Further auxiliaries which may be present in the compositions according to the invention also comprise spreading oils such as di-2-ethylhexyl adipate, isopropyl myristate, dipropylene glycol pelargonate, cyclic and acyclic silicone oils such as dimethicones and also their co- and terpolymers with ethylene oxide, propylene oxide and formalin, fatty acid esters, triglycerides and fatty alcohols.

Antioxidants are sulphites or metabisulphites such as potassium metabisulphite, ascorbic acid, butylhydroxytoluene, butylhydroxyanisole, tocopherol and vitamin E. Preferred antioxidants are butylhydroxytoluene, tocophenol and vitamin E.

Photoprotective agents are e.g. substances from the class of benzophenones or novantisolic acid.

Adhesives are e.g. cellulose derivatives, starch derivatives, polyacrylates, natural polymers such as alginates, gelatin.

The emulsion concentrates according to the invention are prepared by dissolving the active ingredient together with the emulsifier and optionally the coemulsifier and also the other possible additives in the organic solvent. The dissolution can take place at a temperature between room temperature and 100° C., preferably between room temperature and 80° C., further preferably between room temperature and 60° C.

Moreover, the present invention relates to the use of the composition according to the Invention as emulsion concentrate for producing emulsions by incorporating the composition according to the invention into water.

The composition according to the invention is preferably used for producing corresponding emulsions. These emulsions are suitable for the systemic and/or non-systemic control of parasites which occur in animal keeping and animal breeding in domestic animals and livestock, and also zoo animals, laboratory animals, experimental animals and pets. They are effective against all or individual development stages of the pests and also against resistant and normal-sensitivity species of the pests.

Parasites are in particular arthropods, particularly preferably insects. Preference is given to using the preparations according to the invention for controlling ectoparasites.

The aforementioned ectoparasites include: hard ticks, soft ticks, mange mites, leaf mites, flies (biting and licking), parasitic fly larvae, lice, hair lice, feather lice and fleas. These parasites include:

From the order of the Anoplurida, e.g. Haematopinus spp., Linognathus spp., Pediculus spp., Phtirus spp., Solenopotes spp.

From the order of the Mallophagida and the suborders Amblycerina and Ischnocerina. e.g. Trimenopon spp., Menopon spp., Trinoton spp., Bovicola spp., Wemeckiella spp., Lepikentron spp., Trichodectes spp., Felicola spp.

From the order of the Diptera and the suborders Nematocerina and Brachycerina, e.g. Aedes spp., Anopheles spp., Culex spp. Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyla spp., Braula spp., Musca spp., Hydrotaea spp., Stomoxys spp., Haematobia spp., Morellia spp., Fannia spp., Glossina spp., Calliphora spp., Lucilla spp., Chrysomyia spp., Wohlfahrtia spp., Sarcophaga spp., Oestrus spp., Hypoderma spp., Gasterophilus spp. Hippobosca spp., Lipoptena spp., Melophagus spp.

From the order of the Siphonapterida, e.g. Pulex spp., Ctenocephalides spp., Xenopsylla spp. Ceratophyllus spp.

From the order of the Heteropterida, e.g. Cimex spp., Triatoma spp. Rhodnius spp., Panstrongylus spp.

From the order of the Blattarida. e.g. Blatta orientalis, Periplaneta americana. Blattella germanica, Supella spp.

From the subclass of the Acaria (Acarida) and the orders of the meta- and mesostigmata, e.g. Argas spp., Ornithodorus spp., Otobius spp., Ixodes spp. Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp., Varroa spp.

From the order of the Actinedida (Prostigmata) and Acaridida (Astigmata), e.g. Acarapis spp., Cheyletiella spp., Ornithocheyletia spp., Myobia spp., Psorergates spp., Demodex spp., Trombicula spp., Listrophorus spp., Acarus spp. Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active ingredients are also suitable for controlling parasitic protozoa. The parasitic protozoa include:

Mastigophora (Flagellata) such as e.g. Trypanosomatidae, e.g. Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis. L. donovani, L. tropica, such as e.g. Trichomonadidae, e.g. Giardia lamblia, G. canis.

Sarcomastigophora (Rhizopoda) such as Entamoebidae, e.g. Entamoeba histotytica, Hartmanellidae z.B. Acanthamoeba sp. Hartmanella sp.

Apicomplexa (Sporozoa) such as Eimeridae, e.g. Eimeria acervulina, E. adenoides, E. alabahmensis, E. anatis, E. anseris, E. arloingi, E. ashata, E. aubumensis, E. bovis, E. brunetti, E. canis, E. chinchillae, E. clupearum, E. columbae. E. contorta, E. crandalis, E. debliecki. E. dispersa, E. ellipsoidales, E. falciformis. E. faurei, E. flavescens, E. gallopavonis, E. hagani. E. intestinalis, E. iroquoina, E. irresidua, E. labbeana, E. leucarti, E. magna. E. maxima, E. media. E. meleagridis, E. meleagrimitis, E. mitis, E. necatrix, E. ninakohlyakimovae. E. ovis, E. parva, E. pavonis, E. perforans, E. phasani, E. piriformis, E. praecox, E. residua, E. scabra, E. spec., E. stiedal, E. suis, E. tenella, E. truncata, E. truttae, E. zuemii, Globidium spec., Isospora belli, I. canis, I. felis, I. ohioensis, I. rivolta, I. spec., I. suls, Neospara caninum, Neospora hughesi; Cystisospora spec. Cryptosporidium spec., such as Toxoplasmadidae, e.g. Toxoplasma gondii, such as Sarcocystidae, e.g. Sarcocystis bovicanis, S. bovihominis, S. neurona, S. ovicanis, S. ovifelis, S. spec., S. suihominis, such as Leucozoidae, e.g. Leucozytozoon simondi, such as Plasmodiidae, e.g. Plasmodium berghei, P. falciparum, P. malariae, P. ovale, P. vivax, P. spec., such as Piroplasmea, e.g. Babesia argentina, B. bovis, B. canis, B. spec. Theileria parva, Theileria spec. such as Adeleina, e.g. Hepatozoon canis, H. spec.

Also Myxospora and Microspora, e.g. Glugea spec. Nosema spec.

Also Pneumocystis carinii, and Cillophora (Ciliata), such as e.g. Balantidium coli, Ichthiophthirius spec., Trichodina spec., Epistylis spec.

The compounds according to the invention are also effective against protozoa which are found as parasites of insects. Those which may be mentioned are parasites of the strain Microsporida, in particular the genus Nosema. Particular mention may be made of Nosema apis in honeybees.

The livestock and breeding animals include mammals such as, for example, cattle, horses, sheep, pigs, goats, camels, water buffalo, donkeys, rabbits, fallow deer, reindeer; fur-bearing animals such as, for example, mink, chinchilla, racoon; such as e.g. chickens, geese, turkeys, ducks.

Laboratory and experimental animals include mice, rats, guinea pigs, golden hamsters, dogs and cats.

The pets include dogs and cats.

Application may either be prophylactic or therapeutic.

The present invention therefore also relates to corresponding emulsions which are obtained starting from the emulsion concentrates according to the invention in water, and also their application described above.

The present invention is illustrated in more detail by the examples below.

### EXAMPLE 1

### #1415-93

7.5 g of coumaphos and 12.5 g of docusate Na are dissolved in 44.5 g of methyl benzoate with heating to 50-60° C. 30 g of ethyl oleate and 7.5 g of polysorbate 80 (Tween 80) are added. After cooling to 20° C., 100 ml of emulsion concentrate result.

### EXAMPLE 2

### #1415-94

7.5 g of coumaphos and 12.5 g of docusate Na are dissolved in 47.5 g of methyl benzoate with heating to 50-60° C. 30 g of Miglyol 812 and 7.5 g of PEG-35 castor oil (Cremophor EL) are added. After cooling to 20° C., 100 ml of emulsion concentrate result.

### EXAMPLE 3

### #141595

7.5 g of coumaphos and 12.5 g of docusate Na are dissolved in 54.0 g of benzyl benzoate with heating to 50-60° C. 17.5 g of PEG-35 castor oil (Cremophor EL), 5.0 g of n-butanol and 10.0 g of ethyl oleate are added. After cooling to 20° C., 100 ml of emulsion concentrate result.

### EXAMPLE 4

### #1415-96

7.5 g of cyfluthrin and 12.5 g of docusate Na are dissolved in 47.5 g of methyl benzoate with heating to 50-60° C. 30.0 g of medium-chain triglycerides (Miglyol 812) and 7.5 g of PEG-40 castor oil, hydrogenated (Eumulgin HRE 40) are added. After cooling to 20° C., 100 ml of emulsion concentrate result.

### EXAMPLE 5

### #1415-97

7.5 g of cyfluthrin and 12.5 g of docusate Na are dissolved in 44.5 g of methyl benzoate with heating to 50-60° C. 30 g of ethyl oleate and 7.5 g of PEG-15 hydroxystearate (Solutol HS 15) are added. After cooling to 20° C., 100 ml of emulsion concentrate result.

### EXAMPLE 6

### #1415-98

7.5 g of cyfluthrin and 12.5 g of docusate Na are dissolved in 55.0 g of benzyl benzoate with heating to 50-60° C. 5.0 g of n-butanol and 10.0 g of medium-chain triglycerides (Miglyol 812) and 17.5 g of PEG-40 castor oil, hydrogenated (Eumulgin HRE 40) are added. After cooling to 20° C., 100 ml of emulsion concentrate result.

### EXAMPLE 7

### #1415-99

7.5 g of flumethrin and 12.5 g of docusate Na are dissolved in 44.5 g of methyl benzoate with heating to 50-60° C. 30 g of ethyl oleate and 7.5 g of PEG-40 castor oil, hydrogenated (Eumulgin HRE 40) are added. After cooling to 20° C., 100 ml of emulsion concentrate result.

### EXAMPLE 8

### #1415-100

7.5 g of flumethrin and 12.5 g of docusate Na are dissolved in 44.5 g of methyl benzoate with heating to 50-60° C. 30 g of ethyl oleate and 7.5 g of polysorbate 60 (Tween 60) are added. After cooling to 20° C., 100 ml of emulsion concentrate result.

### EXAMPLE 9

### #1415-101

7.5 g of flumethrin and 12.5 g of docusate Na are dissolved in 54.0 g of benzyl benzoate with heating to 50-60° C. 5.0 g of n-butanol, 10.0 g of ethyl oleate and 17.5 g of PEG-35 castor oil (Cremophor EL) are added. After cooling to 20° C., 100 ml of emulsion concentrate result.

1. A composition comprising an ectoparasiticidal active ingredient, at least one anionic surfactant, a nonionic surfactant and an organic solvent.

2. The composition according to claim 1, wherein the ectoparasiticide is selected from the group consisting of phosphoric or phosphonic acid esters, natural or synthetic pyrethroids, carbamates and amidines.

3. The composition according to claim 2, wherein the anionic surfactant is selected from the group consisting of sulphates, sulphosuccinates, alkylarylsulphonic acids and alkylarylsulphonates.

4. The composition according to claim 1, wherein the anionic surfactant is Na dioctylsulphosuccinate.

5. The composition according to claim 3, wherein the organic solvent is selected from the group consisting of benzoic acid esters and limited-miscibility ketones.

6. The composition according to claim 5, wherein the nonionic surfactant is selected from the group consisting of polysorbates, ethoxylated hydroxy fatty acids, ethoxylated castor oils and ethoxylated hydrogenated castor oils.

7. A process for the preparation of the composition of claim 1, characterized in that the constituents of the composition are mixed together.

8. (canceled)

9. (canceled)

10. (canceled)

11. A method of controlling systemic and/or non-systemic parasites in an animal comprising administering the aqueous emulsion according to claim 1 to the animal.
